(19) United States
(12) Patent Application Publication
(10) Pub. No.: US 2026/0001831 A1
(43) Pub. Date: Jan. 1, 2026

(54) COPPER MEDIATED CONVERSION OF ARENES AND ALKENES TO ESTERS, ALCOHOLS, AND ALDEHYDE PRODUCTS

(71) Applicants: William L. Schinski, San Rafael, CA (US); University of Virginia Patent Foundation, Charlottesville, VA (US)

(72) Inventors: Thomas Brent Gunnoe, Palmyra, VA (US); Junqi Chen, Claymont, DE (US); Fanji Kong, Charlottesville, VA (US); William L. Schinski, San Rafael, CA (US)

(21) Appl. No.: 18/877,324

(22) PCT Filed: Jul. 6, 2023
(86) PCT No.: PCT/US2023/069680

§ 371 Date: Dec. 20, 2024

Related U.S. Application Data
(60) Provisional application No. 63/367,901, filed on Jul. 7, 2022.

(51) Int. Cl. C07C 67/04; B01J 31/22; B01J 38/40

(52) U.S. Cl. CPC C07C 67/04; B01J 31/2239; B01J 38/40; C07C 2531/22

(57) ABSTRACT

Described herein are methods for producing an aryl ester or vinyl ester, the method comprising reacting an aromatic compound or an alkene with CuX₂, wherein X is a carboxylate group having the formula RC(O)O—, wherein R is an alkyl group. The methods described herein provide an efficient method for esterifying aromatic compounds and alkenes to produce aryl and vinyl ester that subsequently can be converted to other useful industrial compounds including, but not limited to, alcohols. Also described herein are methods for recycling CuX₂ for further reactions thus rendering CuX₂ as a net catalyst.

### CROSS-REFERENCE TO RELATED APPLICATIONS

This application claims the benefit of and priority to co-pending U.S. Provisional Patent Application No. 63/367,901, filed on Jul. 7, 2022, the contents of which are incorporated by reference herein in their entireties.

### STATEMENT REGARDING FEDERALLY SPONSORED RESEARCH OR DEVELOPMENT

This invention was made with government support under grant number CHE-2102433, awarded by the U.S. National Science Foundation. The government has certain rights in the invention.

### BACKGROUND

As an important industrial chemical, phenol has been deployed in applications in various fields including household products, nylon, polymers, and the manufacturing of other chemical derivatives. Over 12 million tonnes of phenol are produced each year, and greater than 90% of phenol is produced through the Hock process (1-2). However, a major issue with the Hock process is the generation of a stoichiometric amount of acetone as an oversupplied side product (1). Alternative methods for phenol production include the oxidation of benzene using N₂O developed by Solutia, and a three-step route from benzene to phenol with cyclohexanone as a coproduct developed by ExxonMobil (3-5). However, the ExxonMobil method does not provide a desirable coproduct-free route for phenol production, and the Solutia process requires N₂O as oxidant (rather than dioxygen) (FIG. 1). Other related benzene hydroxylation processes using copper rely on the formation of hydroxyl radical and/or require solid-supports to form heterogeneous catalysts (2,6-8), which suffer from low selectivity and/or low yield.

Acetoxylation of benzene has been considered as a potential route for phenol production since hydrolysis of phenyl acetate generates phenol (9). Palladium-catalyzed benzene acetoxylation has been studied (10-11), but only a limited number of examples of aromatic C—H acetoxylation/hydroxylation have been reported using low-cost transition metals, and these examples rely on functionalized aromatic substrates with “directing groups” to facilitate coordination to the catalyst (12-19). Yu and co-workers demonstrated a series of C—H functionalizations using pyridine as the directing group with copper(II) acetate as the catalyst precursor, and the reaction was proposed to occur by the formation of a radical cation intermediate via a single electron transfer (SET) mechanism (12). Later, the Shi group reported a study using a bidentate directing group derived from 2-(pyridine-2-yl) isopropylamine through a proposed Cu(III)-aryl intermediate (13). Subsequently, the Yu group demonstrated a related C—H hydroxylation process using oxazolyamide as the directing group via a possible monomeric Cu(III)-aryl intermediate (15). Benzoic acid has also been reported as a directing group for the synthesis of hydroxylated arenes assisted by benzoyl peroxide (17). Other related C—H functionalization processes by Cu(II) also require a directing group to facilitate the reaction (20-31).

### SUMMARY

In accordance with the purpose(s) of the present disclosure, as embodied and broadly described herein are methods for producing an aryl ester or vinyl ester, the method comprising reacting an aromatic compound or an alkene with CuX₂, wherein X is a carboxylate group having the formula

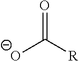

wherein R is an alkyl group. The methods described herein provide an efficient method for esterifying aromatic compounds and alkenes to produce aryl and vinyl ester that subsequently can be converted to other useful industrial compounds including, but not limited to, alcohols. Also described herein are methods for recycling CuX₂ for further reactions thus rendering CuX₂ as a net catalyst.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows current methods for phenol production and the proposed method via Cu(II)-mediated acetoxylation (NHPI=N-hydroxyphthalimide).

FIGS. 2A-2D differentiate between radical and non-radical pathways. (A) Intermolecular competition of sp² vs. sp³ C—H bonds using a mixture of benzene and cyclohexane. N.D.=not detected. (B) Intermolecular competition of sp² vs. sp³ C—H bonds using toluene. (C) meta-Tolyl acetate and benzyl acetate yield versus time plot of reaction using different amount of (2,2,6,6-tetramethylpiperidin-1-yl)oxyl (TEMPO). Reaction conditions: toluene (10 mL, 94.1 mmol), Cu(OAc)₂, (98 mg, 0.540 mmol), TEMPO (0, 0.2, 0.5, and 1.0 equiv. relative to Cu(OAc)₂), 75 psig N₂, 180° C. (D) Observed products of control experiment using TEMPO with no Cu(II) salt. Reaction conditions: toluene (10 mL, 94.1 mmol), TEMPO (71 mg, 0.452 mmol), 75 psig N₂, 170° C. Standard deviations were calculated from at least three independent experiments.

FIGS. 3A-3B show the effects of weakly coordinating (MeO)₃P═O ligands. (A) Sum of tolyl/benzyl 2-ethylhexanoate yields and benzyl 2-ethylhexanoate yield versus time plot with the presence of different amounts of (MeO)₃P═O ligands. Reaction conditions: toluene (10 mL, 94.1 mmol), Cu(OHex)₂, 0.48 mol %, 0.452 mmol), (MeO)₃P═O (0, 0.5, 1.0, and 2.0 equiv. relative to Cu(OHex)₂), 75 psig N₂, 170° C. Standard deviations were calculated from at least three independent experiments. (B) ORTEP of {(MeO)₃P═O}₂Cu₂(μ-OAc)₄. Ellipsoids are drawn at 50% probability level.

FIGS. 4A-4B shows the ligand effects and reaction pathway. (A) Proposed competing pathway in the arene acetoxylation reaction in the presence of weakly coordinating or bidentate ligand. (B) Proposed reaction pathway and associated rate law.

FIGS. 5A-5D provide data from mechanistic studies. (A) Measurements of kinetic isotope effect (KIE) using parallel reactions and intermolecular competition experiments. (B) H/D exchange experiment using acetic acid-d₁ (DOAc) as the deuterium source. (C) Curve fitting of observed reaction rates (rate) versus HOHex concentration plot for 2-ethylhexanoation of toluene using Cu(OHex)₂ with the addition of different amounts of HOHex at 170° C. (D) Eyring plot for toluene functionalization using Cu(OHex)₂ calculated with k_{obs} (in black) and k₁ (in red). Reaction rates were measured for production of tolyl 2-ethylhexanoate over the temperature range 150-180° C. under the conditions with addition of variant amounts of 2-ethylhexanoic acid. Rate equation: rate=k″_{obs}[toluene][Cu dimer]. The rate constant k₁ was calculated using curve fitting of observed reaction rates versus HOHex concentration.

FIGS. 6A-6B show the isolation of products and extension to Cu(II) recycling using dioxygen. (A) Isolation of the phenyl 2-ethylhexanoate/acetate product and recovery of the CuX₂ used in the reaction. Reaction conditions: benzene (20 mL, 224.6 mmol), CuX₂ (0.48 mol % 1.080 mmol, Cu(OAc)₂ 196 mg; Cu(OHex)₂ 378 mg), under 75 psig N₂ at 180° C. for Cu(OAc)₂; 170° C. for Cu(OHex)₂. CuX₂═Cu(OAc)₂ anhydrous, or Cu(OHex)₂. (B) Cu(II)-mediated arene 2-ethylhexanoation with Cu(II) recycling. Reaction conditions: for benzene (10 mL, 112.3 mmol), Cu(OHex)₂ (189 mg, 0.540 mmol), 75 psig N₂, 170° C.; for toluene (10 mL, 94.1 mmol), Cu(OHex)₂ (158 mg, 0.452 mmol), 75 psig N₂, 170° C. Standard deviations were calculated from at least three independent experiments.

FIG. 7 shows the proposed synthetic route for aldehyde production.

FIG. 8 shows the selected results for 1-hexene C—H functionalization reactions using Cu(II)₂-ethylhexanoate.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a solvent,” includes, but are not limited to, mixtures or combinations of two or more such solvents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

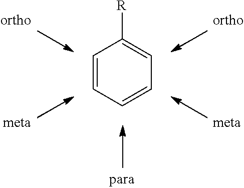

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂-(methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as-OA¹ where A¹ is alkyl or cycloalkyl as defined above. “Alkoxy” also includes polymers of alkoxy groups as just described; that is, an alkoxy can be a polyether such as-OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²) C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having delocalized IT electrons for which where the π system contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “aromatic compound” as used herein is a compound possessing one or more aryl groups as defined herein.

The term “alkene” as used herein is a hydrocarbon compound of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkene can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “terminal alkene” as used herein is a hydrocarbon compound of from 3 to 24 carbon atoms with a structural formula containing at least one carbon-carbon double bond as represented by Y—CH═CH₂, where Y can be, for example, a substituted or unsubstituted alkyl group as defined herein.

The term “aryl ester” as used herein is represented by the structure

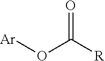

where Ar is an aryl group as defined herein and R can be, for example, an alkyl group as defined herein.

The term “vinyl ester” as used herein is represented by the structure

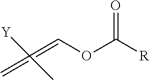

where Y is an alkyl group as defined herein positioned at either carbon atom of the carbon-carbon double bond and each R can be, for example, an alkyl group as defined herein.

The term “terminal vinyl ester” as used herein is represented by the structure

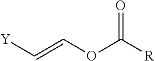

where Y is an alkyl group as defined herein and each R can be, for example, an alkyl group as defined herein.

The term “inert atmosphere” as used herein is an atmosphere composed of an inert gas that is substantially free of oxygen. Examples of inert gases include, but are not limited to, nitrogen or argon. In one aspect, the inert atmosphere has less than 10 ppm of oxygen, less than 5 ppm of oxygen, less than 2 ppm of oxygen, or no detectable oxygen.

The term “anhydrous” as used herein is a substance, compound, solvent, or atmosphere that is substantially fee of water. In one aspect, anhydrous is a substance that has less than 5 ppm of water, less than 3 ppm of water, less than 1 ppm of water, or no detectable water.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, BF, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Methods for Producing Aryl and Vinyl Esters

Described herein are methods for producing an aryl ester or vinyl ester from aromatic compounds and alkenes, respectively. The methods described herein provide an efficient method for esterifying aromatic compounds and alkenes to produce aryl and vinyl ester that subsequently can be converted to other useful industrial compounds.

The method comprises reacting an aromatic compound or an alkene with CuX₂, wherein X is a carboxylate group having the formula I

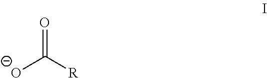

wherein R is an alkyl group.

In one aspect, the aromatic compound or alkene are reacted with CuX₂ under an inert atmosphere, where the reaction is conducted under an elevated temperature. In one aspect, the reaction temperature is from 150° C. to about 300° C., or about 150° C., 175° C., 200° C., 225° C., 250° C., 275° C., or 300° C., where any value can be a lower and upper endpoint of a range (e.g., 175° C. to 200° C.). The Examples provide non-limiting procedures for conducting the methods described herein.

The aromatic compound or alkene and CuX₂ can be admixed with one another for a time sufficient to complete the reaction using techniques known in the art. The reaction can be monitored by techniques known in the art such as gas chromatography and/or mass spectroscopy. The Examples provide non-limiting techniques for monitoring the reactions described herein as well as reaction products.

The copper compound CuX₂ used in the reactions described herein can vary depending upon the aryl or vinyl ester to be produced. In one aspect, R in formula I is a branched or straight chain C₁ to C₂₀ alkyl group. In one aspect, R in formula I is —CH₃, —CH(CH₃)₂, —C(CH₃)₃, or —(CH₂)₅CH₃ or 2-ethylthexanoate.

The amount of the aromatic compound or alkene and CuX₂ can vary depending upon reaction conditions as well as the selection of the aromatic compound or alkene. In one aspect, CuX₂ is from about 0.1 mol % to about 10.0 mol % relative to the molar amount of the aromatic compound or alkene, wherein the sum of the molar amount of the aromatic compound or alkene and CuX₂ is 100 mol %, or CuX₂ is about 0.1 mol %, 0.2 mol %, 0.3 mol %, 0.4 mol %, 0.5 mol %, 0.6 mol %, 0.7 mol %, 0.8 mol %, 0.9 mol %, 1.0 mol %, 2.0 mol %, 3.0 mol %, 4.0 mol %, 5.0 mol %, 6.0 mol %, 7.0 mol %, 8.0 mol %, 9.0 mol %, or 10.0 mol %, relative to the molar amount of the aromatic compound or alkene, where any value can be a lower and upper endpoint of a range (e.g., 0.3 mol % to 0.7 mol %).

In certain aspects, the aromatic compound, alkene, and CuX₂ can be processed further prior to conducting the reactions described herein. In one aspect, the aromatic compound and alkene can be further dried to produce anhydrous materials. For example, when the aromatic compound or alkene is a liquid, the liquid can be distilled, including from a drying agent, to purify the aromatic compound or alkene and separate any impurities such as, for example, water. In another embodiment, the aromatic compound or alkene when a liquid can be contacted with molecular sieves to remove any residual water. In another aspect, when the aromatic compound or alkene is a solid, the solid can be heated for a sufficient time and temperature to remove water residual water from the solid.

In other aspect, solid CuX₂ be heated for a sufficient time and temperature to remove water residual water from the solid to produce anhydrous CuX₂. In other aspects, solid CuX₂ be crushed or grinded into smaller particles to enhance the performance of CuX₂ in the reactions described herein.

In the case when the aromatic compound or alkene are solid compounds, an organic solvent can be used to solubilize the compounds as well as CuX₂. In one aspect, the solvent is an inert solvent that does not react with CuX₂. In another aspect, the solvent is an anhydrous organic solvent.

The methods described herein provide an efficient approach to producing aryl esters. The aryl esters produced herein can ultimately be converted to other useful commercial products. For example, when the aromatic compound is benzene, the aryl ester produced by the method described herein can be a precursor to the production of phenol as provided below.

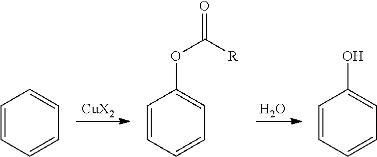

In the case when the aromatic compound is a substituted compound, for example when the aromatic compound is toluene, the ester group can be present at the ortho, meta, and para positions as depicted below.

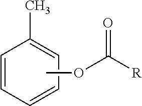

In the case when multiple aryl esters are produced (i.e., ortho, meta, or para aryl esters), each aryl ester can be separated using techniques known in the art.

In the case when the aromatic compound is substituted, the compound can be substituted with one or more alkyl groups. In one aspect, the aromatic compound is benzene substituted with one or more alkyl groups.

The methods described herein provide an efficient approach to producing vinyl esters. The vinyl esters produced herein can ultimately be converted to other useful commercial products. For example, when the vinyl ester produced by the method described is a terminal vinyl ester, the terminal vinyl ester herein can be converted to aldehydes as provided below.

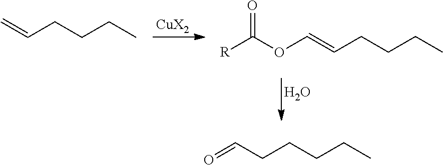

Also described herein are methods for recycling CuX₂ for further reactions. After the aryl or vinyl ester is produced, CuX is produced as well. In one aspect, the corresponding carboxylic acid can be added to CuX in the presence of air to produce CuX₂. This reaction is depicted below.

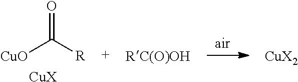

In the reaction above, R and R′ are the same group (e.g., CH₃). After the reaction, CuX² can be isolated and reused in subsequent reactions. Water is also produced in the reaction, which can be removed using techniques known in the art. The Examples provide nonlimiting procedures for reproducing and recycling CuX₂.

### Aspects

The present disclosure can be described in accordance with the following numbered aspects, which should not be confused with the claims.
Aspect 1. A method for producing an aryl ester or a vinyl ester, the method comprising reacting an aromatic compound with CuX₂ to produce the aryl ester or reacting an alkene with CuX₂ to produce the vinyl ester, wherein X is a carboxylate group having the formula

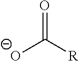

wherein R is an alkyl group.
Aspect 2. The method of Aspect 1, wherein the aromatic compound is benzene or substituted benzene.
Aspect 3. The method of Aspect 1, wherein the aromatic compound is benzene substituted with one or more alkyl groups.
Aspect 4. The method of Aspect 1, wherein the alkene is a terminal alkene.
Aspect 5. The method of Aspect 1, wherein the alkene is a branched or straight chain C₂ to C₂₄ alkene.
Aspect 6. The method of Aspect 1, wherein the vinyl ester is a terminal vinyl ester.
Aspect 7. The method in any one of Aspects 1-6, wherein R is a branched or straight chain C₁ to C₂₀ alkyl group.
Aspect 8. The method in any one of Aspects 1-7, wherein R is —CH₃, —CH(CH₃)₂, —C(CH₃)₃, or —(CH₂)₅CH₃ or 2-ethylthexanoate.
Aspect 9. The method in any one of Aspects 1-8, wherein the reaction is conducted under an inert atmosphere.
Aspect 10. The method in any one of Aspects 1-9, wherein CuX₂ is from about 0.1 mol % to about 10.0 mol % relative to the molar amount of the aromatic compound or alkene, wherein the sum of the molar amount of the aromatic compound or alkene and CuX₂ is 100 mol %.
Aspect 11. The method in any one of Aspects 1-10, wherein the reaction comprises admixing the aromatic compound or alkene with CuX₂ at a temperature of from about 150° C. to about 300° C.
Aspect 12. The method in any one of Aspects 1-11, wherein CuX₂ is anhydrous.
Aspect 13. The method in any one of Aspects 1-12, wherein the reaction is conducted in an organic solvent.
Aspect 14. The method of Aspect 13, wherein the organic solvent is an anhydrous solvent.
Aspect 15. The method in any one of Aspects 1-14, wherein after the reaction CuX is produced, wherein CuX is converted to CuX₂ by (1) adding RC(O)OH (or using RC(O)OH generated in situ) in the presence of oxygen to the reaction to produce CuX₂ and water, wherein R is the same alkyl group as CuX₂, and (2) removing water from the reaction.
Aspect 16. The method of Aspect 15, wherein water is removed by molecular sieves or distillation.
Aspect 17. The method of Aspect 15 or 16, wherein the recycled CuX₂ is reintroduced into the reaction with the aromatic compound or alkene.

### Examples

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Materials and Methods

General Considerations. All reactions were performed under a dinitrogen atmosphere using Schlenk line techniques or inside a dinitrogen filled glovebox unless specified otherwise. GC-FID was performed using a Shimadzu GC-2014 system with a 30 m×0.32 mm DB-5 ms UI capillary column with 0.25 μm film thickness. GC-MS was performed using a Shimadzu GCMS-QP2010 Plus or a Shimadzu GCMS-QP2020 NX with a 30 m×0.25 mm capillary column with Rxi-5 ms with 0.25 μm film thickness using electron impact ionization method. Toluene was dried using sodium-benzophenone/ketyl stills under dinitrogen atmosphere and stored inside the glovebox. Methanol was dried using a calcium hydride still under dinitrogen atmosphere and stored inside a glovebox. Benzene, acetonitrile, and methylene chloride were dried using a solvent purification system with activated alumina. Toluene-d₈, acetonitrile-d₃, benzene-d₆, and methylene chloride-d₂ were dried and stored over activated 3 Å molecular sieves inside a glovebox. Except for the Cu(II) salts (see below), all other chemicals were purchased from commercial sources and used as received. Elemental analyses were performed by the University of Virginia Chemistry Department Elemental Analysis Facility.

General Procedures for Pretreatment of Cu(II) Salts. The commercial Cu(II) salts was ground and heated in a Schlenk flask under ˜10 mTorr vacuum in a 110° C. oil bath for at least 1 day or 90° C. for 3 days. During the drying process, the condensation of a clear liquid was observed on the side of the round bottom flask, and a heat gun was used to completely transfer the liquid into the liquid nitrogen trap on the Schlenk line. After drying, the needle valve was sealed and the flask containing Cu(II) salts was directly transferred into the glovebox without opening to air.

Alternate method to purify Cu(OHex)₂. Commercial Cu(OHex)₂ (10 g) was dissolved in 60 mL of EtOH and stirred until forming a homogeneous solution. Then deionized water was added until no more precipitation stopped. The mixture was filtered to collect the fine greenish-blue solid. After washing with water a few times, the solid was placed in a vacuum oven at 100° C. until no mass change to yield the final product (9.3 g). This method particularly useful for removing high boiling point 2-ehtylhexanoic acid in the commercial Cu(OHex)₂.

Synthesis of Copper (II)₂-ethylhexanoate (Cu(OHex)₂). To a solution of NaOH (10 g, 250 mmol) in 300 mL of EtOH, 2-ethylhexanoate acid (42 mL, 263 mmol) was added and stirred for 2 hours at room temperature. Then, Cu(NO₃)₂·3H₂O (30.2 g, 125 mmol) was added to the solution. The color of the solution was blue and then changed to a greenish-blue color with solid precipitation. The mixture was then stirred at room temperature for at least 2 hours, then deionized water was added to precipitate out the solid product as a greenish-blue fine solid. The solid was washed with deionized water to remove the water-soluble salts. Then the solid product was placed in a vacuum oven heated to 100-110° C. under vacuum until no mass changes to yield the final Cu(OHex)₂ product (39.4 g, 90% isolated yield). The solid can be further dried using the same procedure described above but can also be used directly without further drying process.

Synthesis of (MeO)₃P═O Cu(OAc)₂. To a solution of Cu(OAc)₂ (100 mg, 0.551 mmol) in 5 mL of toluene, trimethyl phosphate (1 mL, 8.639 mmol) and 1 mL of HOAc were added. After heating at reflux for approximately 1 hour, the suspension was filtered hot to remove the undissolved Cu(OAc)₂ (most of the Cu(OAc)₂ was not dissolved), and crystals suitable for X-ray analysis were obtained by slow evaporation from light-blue filtrate (20 mg, 11% isolated yield). Anal. Calcd for C₁₄H₃₀O₁₆P₂Cu₂: C, 26.13; H, 4.70. Found: trial #1 C, 25.84; H, 4.48.; trial #2 C, 25.92; H, 4.49.

Synthesis of (MeO)₃P═O Cu(OPiv)₂. To a solution of Cu(OPiv)₂ (100 mg, 0.389 mmol) in 5 mL of toluene, trimethyl phosphate (45 μL, 0.389 mmol) was added. After heating at reflux for approximately 1 hour, the solution was filtered hot, and crystals suitable for X-ray analysis were obtained from slow evaporation from the blue filtrate (82 mg, 53% isolated yield). Anal. Calcd for C₂₆H₅₄P₂O₁₆Cu₂: C, 38.47; H, 6.71. Found: C, 38.53; H, 6.96.

General procedure for Cu(II)-mediated arene C—H acetoxylation. Under an atmosphere of dry nitrogen inside a glovebox that the purity was maintained as O₂<5 ppm, and H₂O<1 ppm, the copper (II) salt {CuX₂, X═OAc, 0.540 mmol (0.48 mol % to relative to benzene, 0.57 mol % relative to toluene); for soluble CuX₂, X=OPiv or OHex, 0.48 mol % relative to arene (0.540 mmol for reaction using benzene; 0.452 mmol for reaction using toluene)} was added into a dried Andrews Glass Lab-Crest Fisher-Porter tube with a stir bar, then 10 mL of arene (benzene or toluene) was measured by a syringe and added to the reaction tube. The Fisher-Porter tube was then sealed and pressurized with 75 psig of nitrogen. Then the mixture was stirred in an oil bath at reaction temperature (the oil bath temperature was confirmed by an external mercury thermometer), and the stir plate stirring speed was set to 550 rpm. The reaction time was recorded starting at the time the oil bath temperature reached the setting point, which typically required 5-10 mins after placing the Fisher-Porter tube into the 1 L oil bath with 500-600 mL of silicone oil (usable range from −40 to 200° C.). The reaction was monitored periodically by taking a 100 μL aliquot of the reaction solution and mixing with 100 μL of a hexamethylbenzene (HMB) stock solution in arene (benzene or toluene) with known HMB concentration, then quickly washing the mixture with saturated sodium hydroxide solution (˜0.2 mL). The resulting organic layer was subjected to GC-FID and/or GC-MS for quantitative analysis, using relative peak areas versus HMB as the standard.

### Results

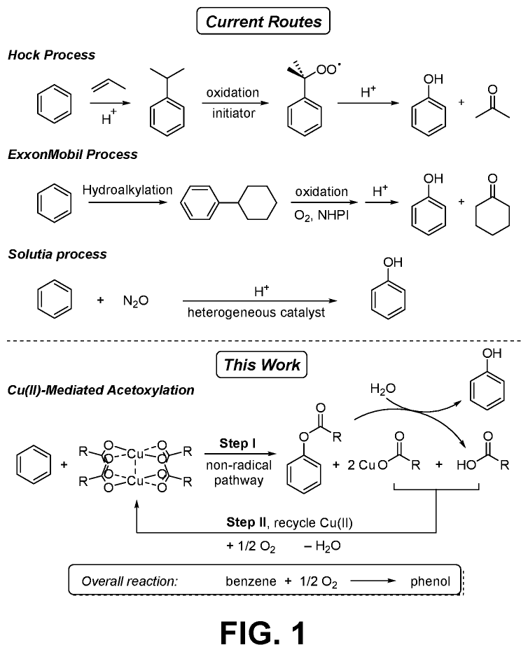

To our knowledge there are no reported examples of molecular Cu(II)-mediated arene acetoxylation without directing groups on the arene. Herein, we report acetoxylation of benzene and toluene using simple Cu(II) salts, and we propose that the C—H functionalization occurs by a non-radical organometallic pathway that forms Cu-aryl intermediates under certain conditions. The Cu(II) salt offers recyclability using O₂ or air, similar to the commercialized Pd-catalyzed ethylene oxidation Wacker process that has proven viable with Cu(II) recycling both in situ (with purified O₂) and in a separate step/reactor from the ethylene oxidation process (using air) (32-35). This process can potentially lead to a coproduct-free synthetic route for phenol production (FIG. 1), since all of the coproducts are used for recycling of the Cu(II) salt, and thus the overall reaction is the conversion of benzene and dioxygen to phenol (FIG. 1).

Development of non-directed Cu(II)-mediated C—H acetoxylation of benzene. The formation of phenyl acetate formation is a result of a reaction of Cu(OAc)₂ with benzene (36). Heating 10 mL of a benzene mixture with pre-dried Cu(OAc)₂ (0.48 mol % relative to benzene) at 180° C. under nitrogen (75 psig) produces phenyl acetate in 87(1) % yield based on the Cu(II) limiting reagent as determined by GC-FID (Table 1, entry 2). The control experiment using CuCl₂ showed no activity for C—H activation on benzene (Table 1, entry 12), which likely indicates the carboxylate group is essential for arene C—H activation (see below). Similar to Cu(OAc)₂, more soluble Cu(II) salts, such as Cu(II) pivalate (Cu(OPiv)₂) and Cu(II) 2-ethylhexanoate (Cu(OHex)₂), were shown to achieve similar reactions at even a lower temperature (Table 1, entries 6, 7 and 9). The Cu(II) species Cu(OPiv)₂ was found to give a lower yield compared to the other Cu(II) salts, which we attribute to the lower thermostability of the pivalic group. The presence of air was found to inhibit the reaction (Table 1, entry 4), and the reaction was slowed down with the addition of an increasing amount of carboxylic acid (Table 1, entries 3, 5, 8, 10, 11). Different ligands and solvents were tested for the reaction; however, none of them provided a beneficial effect. These observations are distinct from the previously published directed Cu(II)-mediated acetoxylation processes (12,14-19), in which the presence of acid, air or ligands are commonly required by the reactions.

TABLE 1
Selected results for benzene acetoxylation and related reactions using
Cu(II) salts. Yield of the phenyl carboxylate is relative to the limiting reagent (CuX₂) as determined
by GC-FID. The theoretical maximum conversion of benzene will be 0.24%. Reaction conditions:
benzene (10 mL, 112.3 mmol), CuX₂ (0.48 mol %, 0.540 mmol), 75 psig N₂. CuX₂ = Cu(OAc)₂
anhydrous; Cu(OPiv)₂, or Cu(OHex)₂. N.D. = not detected. Standard deviations were calculated
from at least three independent experiments.

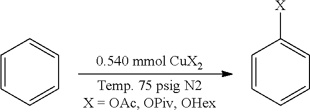

| | | Temp. | HX | | Yield | Time |
|---|---|---|---|---|---|---|
| Entry | Cu(II) salt | (° C.) | (equiv.) | Air | (%) | (h) |
| 1 | Cu(OAc)₂ | 180 | 0 | none | 78(0) | 24 |
| 2 | Cu(OAc)₂ | 180 | 0 | none | 87(1) | 40 |
| 3 | Cu(OAc)₂ | 180 | 1.0 | none | 80(1) | 40 |
| 4^{a} | Cu(OAc)₂ | 180 | 0 | 1 atm. | 15(2) | 24 |
| 5 | Cu(OAc)₂ | 180 | 4.0 | none | 65(6) | 48 |
| 6 | Cu(OPiv)₂ | 180 | 0 | none | 49(1) | 16 |
| 7 | Cu(OPiv)₂ | 170 | 0 | none | 52(0) | 16 |
| 8 | Cu(OPiv)₂ | 170 | 1.0 | none | 36(1) | 16 |
| 9 | Cu(OHex)₂ | 170 | 0 | none | 89(1) | 24 |
| 10 | Cu(OHex)₂ | 170 | 1.0 | none | 71(1) | 40 |
| 11 | Cu(OHex)₂ | 170 | 4.0 | none | 37(0) | 72 |
| 12^{b} | CuCl₂ | 180 | 0 | none | N.D. | 24 |

^{a}Using 1 atm of air, the total top pressure of air and N₂ is 75 psig.
^{b}Using half the amount of reagents in a VCO reactor under 100 psig Ar.

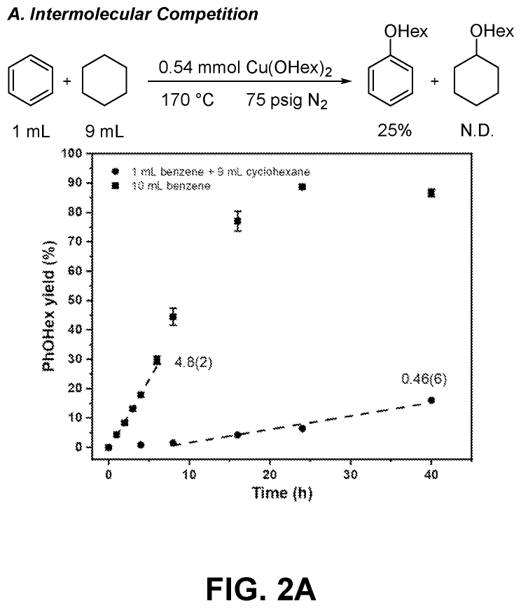

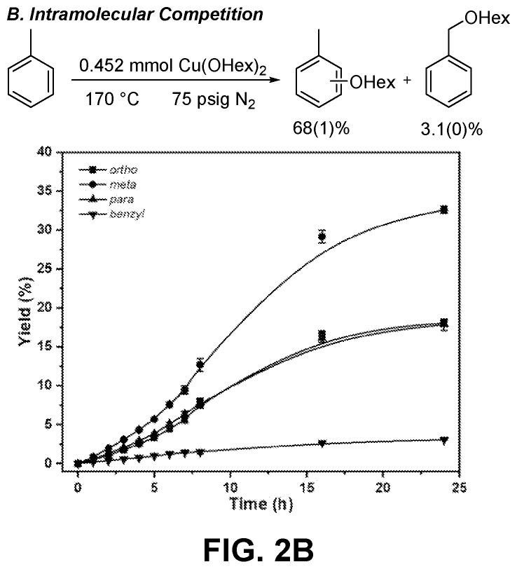

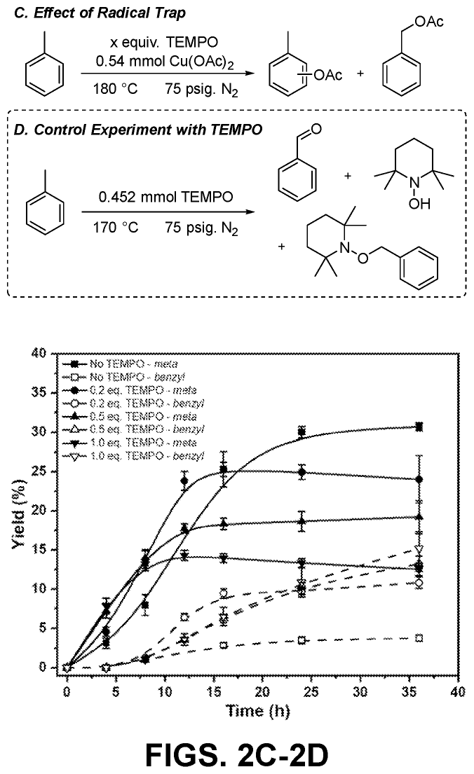

Mechanistic studies. To differentiate between radical and non-radical pathways, separate reactions of toluene or a mixture of benzene with cyclohexane were used as probes. These reactions offer intramolecular and intermolecular competition of weaker sp³ C—H bonds and stronger sp² C—H bonds (FIGS. 2A and B). It is expected that a radical C—H abstraction reaction will select for the weaker sp³ C—H bonds. Under anhydrous conditions, all of the reactions were found to select for the stronger sp² C—H bonds, which suggests that the reactions likely undergo a non-radical reaction pathway. Using 1 mL of benzene with 9 mL of cyclohexane resulted in ˜10% of the rate compared to the reaction using neat benzene, consistent with the reaction being first-order in benzene. A common radical trap, (2,2,6,6-tetramethylpiperidin-1-yl)oxyl (TEMPO), was tested as an additive in the reaction of toluene with Cu(OAc)₂ (FIG. 2C). The reactions with TEMPO were slow and remained selective for tolyl acetates, but more benzyl acetate was formed compared to reactions without TEMPO. In a control experiment, we found that TEMPO can functionalize the benzylic position of toluene without Cu(II) salt (FIG. 2D). When using toluene as the substrate, the regioselectivity of the sp² C—H bonds can be used to differentiate between a potential electrophilic aromatic substitution mechanism (ArSE, with no M-C bond formation), and a metal-mediated C—H activation mechanism via M-C bond formation (37-38). The Cu(II) mediated reaction was found to be more selective for the meta-tolyl carboxylate than the ortho-tolyl carboxylate (Table 2, entries 1, 4 and 5), which we propose (see below) is a result of an organometallic reaction that forms Cu-tolyl intermediates. We propose that the Cu-mediated toluene C—H activation favors the meta-over the ortho-position of toluene due to the steric effect of the methyl group.

In the presence of 1 atm of dried O₂, the selectivity of toluene acetoxylation reaction using Cu(OAc)₂ changed to form benzyl acetate (Table 2, entry 3), which is the evidence for a radical process since the weaker benzyl C—H bond was functionalized. Also, the inclusion of O₂ suppresses reactivity with the stronger sp² tolyl C—H bonds, similar to our observation when using benzene as the substrate that exhibited lower yield in the presence of O₂(Table 1, entry 4). The selectivity shifts from sp² C—H bond functionalization to benzylic functionalization is expected since mixing Cu(II) with O₂ is known to form Cu-peroxo and/or superoxo species (39-42), which likely leads to the formation of free radical species that favor weaker sp³ C—H bond functionalization over stronger sp² C—H bonds. Since hexamethylbenzene (HMB), which has been used as the standard for GC analysis, contains benzylic C—H bonds and the Cu(II) mediated functionalization of toluene activates benzyl C—H bonds under some conditions, control experiments were done using Cu(OAc)₂ and toluene as the substrate at 180° C. with in situ and ex situ addition of HMB for GC-FID analysis. As expected, under anaerobic conditions, the consumption of HMB is negligible, while under aerobic conditions a competing radical-based reaction with HMB was observed, which is consistent with our proposal that reaction pathways are distinct under anaerobic versus aerobic conditions. Therefore, to avoid complications caused by the consumption of HMB, all the reported quantifications used ex situ addition of HMB for GC analysis. Water was also examined as an additive (Table 2, entries 4 and 11), and similar to O₂, the presence of 1 equivalent of water (relative to Cu) switches the reaction selectivity for toluene activation towards benzylic C—H functionalization although tolyl products are still observed. We suspect that water coordinates to Cu₂(μ-OAc)₄ to form copper (II) acetate hydrate, Cu₂(μ-OAc)₄·2H₂O, which can convert to monomeric Cu(OAc)₂·(H₂O)ₙ (43) or other decomposition species such as Cu/Cu₂O under reaction temperatures that could lead to a radical process(es) that competes with the organometallic Cu(II)-mediated pathway. The observation of a similar change in reaction selectivity using commercial copper(II) acetate monohydrate [Cu₂(μ-OAc)₄·2H₂O] (Table 2, entry 5) is consistent with the observation of reactions with water added to anhydrous Cu₂(μ-OAc)₄. The addition of increasing amounts of carboxylic acid (Table 2, entries 6 and 12-14) decreases the reaction rate without changing the benzyl-to-tolyl selectivity. This suggests that carboxylic acid inhibits the organometallic reaction pathway but does not facilitate undesirable radical-based processes, which is different from the effect of water or O₂.

TABLE 2
Selected results for toluene acetoxylation and related reactions using
simple (Cu(II) salts. Yield of the tolyl or benzyl carboxylate is relative to the limiting reagent
(CuX₂). Reaction conditions: toluene (10 mL, 94.1 mmol), CuX₂ (for Cu(OAc)₂, 0.57 mol %, 0.540
mmol; for Cu(OPiv)₂ or Cu(OHex)₂, 0.48 mol %, 0.452 mmol), 75 psig N₂. CuX₂ = Cu(OAc)₂
anhydrous, Cu(OPiv)₂, or Cu(OHex)₂. Standard deviations were calculated from at least three
independent experiments.

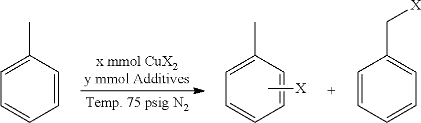

| | | Additive | Yield (%) | | | | |
|---|---|---|---|---|---|---|---|
| Entry | CuX₂ | (equiv.) | Total | Benzyl | Para | Meta | Ortho |
| 1 | Cu(OAc)₂ | none | 72(1) | 4.0(5) | 19(0) | 31(1) | 18(0) |
| 2 | Cu(OAc)₂ | none | 71(2) | 5.0(2) | 20(1) | 31(1) | 15(1) |
| 3^{a} | Cu(OAc)₂ | Dried O₂ | 95(6) | 89(6) | 1(0) | 3(0) | 2(0) |
| 4 | Cu(OAc)₂ | H₂O (1.0) | 37(2) | 17(2) | 5(0) | 10(0) | 5(0) |
| 5 | Cu(OAc)₂•H₂O | none | 48(3) | 23(0) | 7(1) | 13(2) | 6(1) |
| 6 | Cu(OAc)₂ | HOAc (1.0) | 50(3) | 3.0(3) | 12(1) | 23(2) | 11(1) |
| 7 | Cu(OAc)₂ | Ac₂O (1.0) | 3(1) | 0.3(0) | 1(0) | 2(0) | 1(0) |
| 8 | Cu(OPiv)₂ | none | 33(0) | 0.9(1) | 10(0) | 16(0) | 7(0) |
| 9 | Cu(OHex)₂ | none | 54(1) | 2.4(2) | 16(0) | 24(0) | 12(0) |
| 10 | Cu(OHex)₂ | none | 72(0) | 3.1(1) | 18(1) | 33(1) | 18(0) |
| 11 | Cu(OHex)₂ | H₂O (1.0) | 41(3) | 8.4(0) | 9(1) | 16(2) | 7(1) |
| 12 | Cu(OHex)₂ | HOHex (1.0) | 68(2) | 2.9(1) | 19(1) | 33(1) | 13(0) |
| 13 | Cu(OHex)₂ | HOHex (2.0) | 56(1) | 2.8(3) | 15(0) | 28(1) | 10(3) |
| 14 | Cu(OHex)₂ | HOHex (4.0) | 36(1) | 2.3(2) | 10(0) | 19(1) | 6(1) |

^{a}Using 1 atm of dried O₂, the total top pressure of O₂ and N₂ is 75 psig.

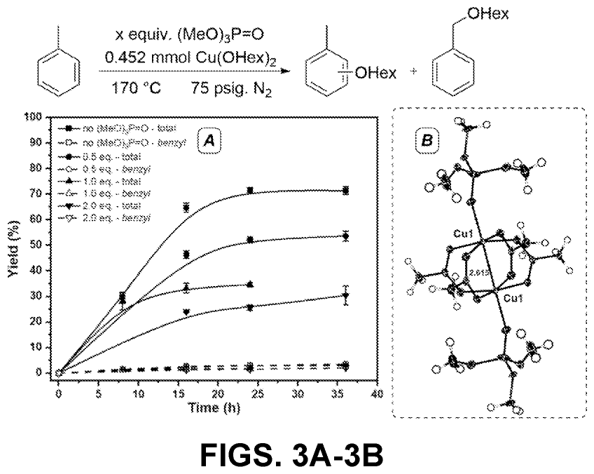

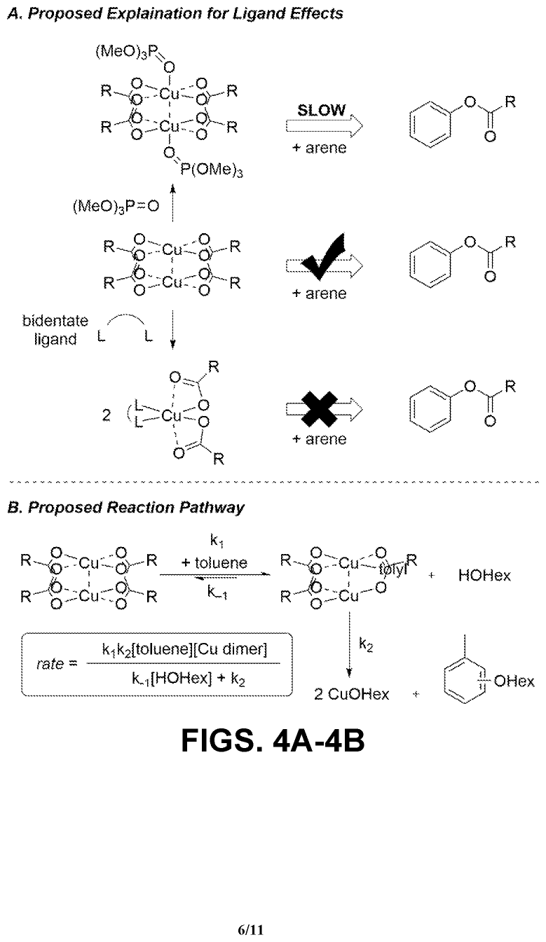

The presence of bi- or tridentate ligand shuts down the benzene functionalization reaction, and many ligands result in a switch of the reaction selectivity for toluene activation towards benzylic C—H functionalization. However, using (MeO)₃P═O, which we presume is a weakly coordinating ligand, only slows down the arene functionalization process without changing the sp²-to-sp³ selectivity (FIG. 3A). By refluxing Cu(OAc)₂ and (MeO)₃P═O in toluene, the resulting product was found to be the bis-Cu complex {(MeO)₃P═O}₂Cu₂(μ-OAc)₄. The structure of {(MeO)₃P═O}₂Cu₂(μ-OAc)₄ was confirmed by a single crystal X-ray diffraction study (FIG. 3B). We speculate that (MeO)₃P═O does not disrupt the dimeric Cu(II) structure and, hence, only slows down the functionalization of arenes by competing with the arene substrate for Cu coordination. In contrast, multi-dentate ligands likely convert bis-Cu(II) to monomeric Cu(II) complexes of the type LCu^{II}X₂ that are not active for the functionalization of arenes (FIG. 4A). By monitoring the initial rate of the reaction using different amounts of soluble Cu₂(μ-OHex)₄ and 2-ethylhexanoic acid (HOHex), the reaction was found to be first-order in Cu₂(μ-OHex)₄ concentration and inverse first-order in HOHex concentration. Based on these observations, we propose a plausible reaction pathway and rate equation (FIG. 4B). Reversible arene C—H activation by Cu₂(μ-X)₄ (X═OAc, OHex or OPiv) and dissociation of carboxylic acid is followed by an overall rate limiting reductive elimination of aryl ester.

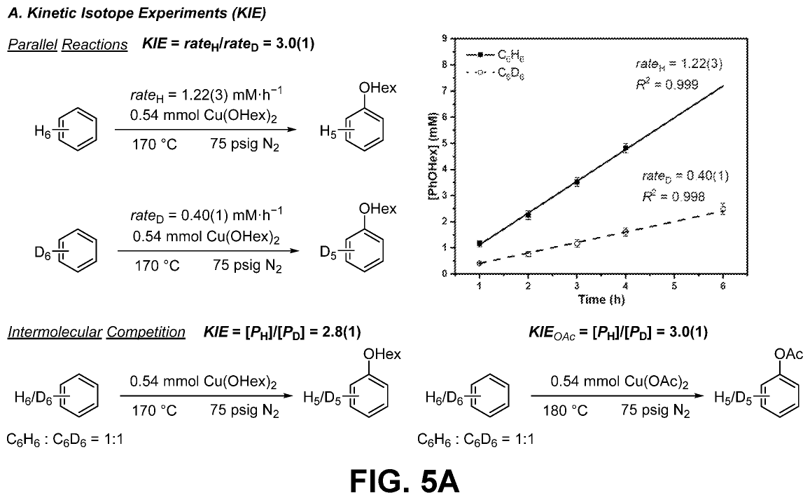

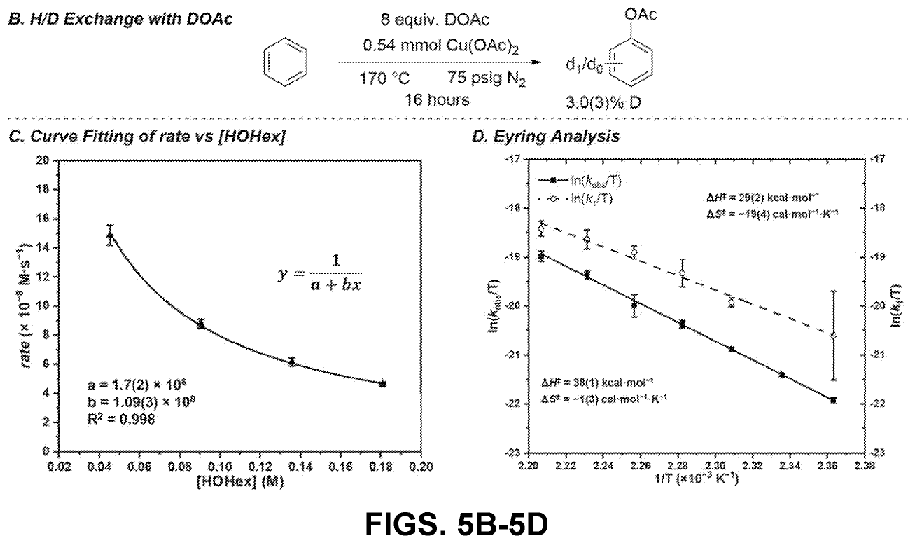

Based on the proposed rate equation (FIG. 5B), the rate constant for the C—H activation step (k₁) can be calculated via a curve fitting of rate versus [HOHex] (FIG. 5C) or the intercept from a 1/rate versus [HOHex] plot, where rate is the slope of the concentration of tolyl 2-ethylhexanoate versus time plot. Both plots gave good fits, which is consistent with the proposed reaction pathway. The rate constant for the C—H activation step (k₁) and the related activation free energy (ΔG₁^{‡}_{443K}) can be estimated from the intercept as 2.7 (4) 10⁻⁶ s⁻¹·M⁻¹ and 38(1) kcal·mol⁻¹. A slight induction period was observed for all of the Cu(II)-mediated acetoxylation reactions without addition of carboxylic acid. The reaction rate of the induction period and the initial linear region were monitored at different reaction temperatures using soluble Cu(OHex)₂ with toluene as the substrate. It was found that a decrease in reaction temperature led to an increase in the induction period, and at 180° C., there is no observable induction period. The induction period of the reaction was also studied in the presence of variable concentrations of HOHex, and with the addition of more acid, the induction period becomes less obvious. We propose that the induction period is due to the process of dissolving Cu(II) salt under acid free conditions.

To gain insight into the Cu(II)-mediated arene C—H activation step, we studied the kinetic isotope effect (KIE) using C₆H₆ and C₆D₆ as the substates with soluble Cu(OHex)₂ at 170° C. The KIE value was determined to be 3.0(1) for parallel reactions using benzene and benzene-d₆, and 2.8(1) for the intermolecular competition reactions using a mixture of benzene and benzene-d₆ with a 1-to-1 molar ratio (FIG. 5A). This type of KIE has typically been interpreted as C—H cleavage that occurs during or before the rate-determining step (44-45), which is consistent with the proposed reaction pathway (FIG. 4B). In the presence of 8 equivalents of DOAc (relevant to Cu(OAc)₂), a small amount of deuterium incorporation (3.0 (3) %) was found in the product (FIG. 5B), which likely indicates that the C—H activation step is partially reversible and is also consistent with the proposed reaction pathway (FIG. 5B).

An Eyring analysis was performed using reaction rates measured at seven temperatures between 15° and 180° C. (FIG. 5D, solid line), which provided an activation enthalpy (ΔH^{‡}) of 38(1) kcal·mol⁻¹. The activation entropy (ΔS^{‡}) was found to be −1(3) cal·mol⁻¹·K⁻¹ assuming an overall second order rate law (rate=k″_{obs}[toluene][Cu₂(μ-OHex)₄]), which indicates that the entropy has a negligible effect on the overall reaction. The small entropy of activation is potentially due to the use of excess toluene as the solvent and/or that the C—H activation step may only be partially rate limiting. Using an Arrhenius plot, the activation energy (Ea) was determined to be 39(1) kcal·mol⁻¹. As described above, the rate constant of the C—H activation step (k₁) can be determined, therefore, a set of reaction have been done over the temperature range 150-180° C. under the conditions with addition of variant amounts of 2-ethylhexanoic acid in order to calculate k₁ at different temperatures. The Eyring analysis performed using k₁ at different temperatures as it shown in FIG. 5D (dash line), and these data indicate a ΔH^{‡} of 29(2) kcal·mol⁻¹ and a ΔS^{‡} of −19(4) cal·mol⁻¹·K⁻¹.

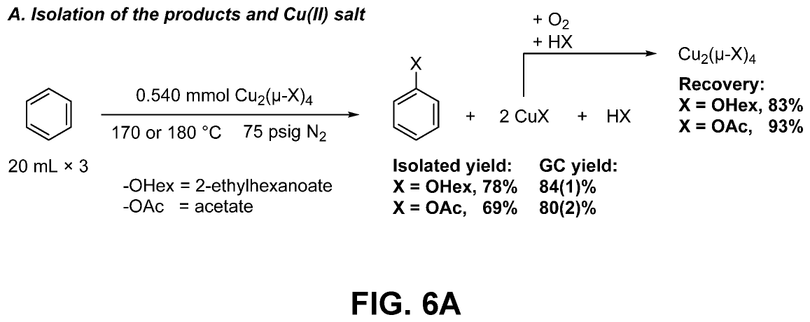

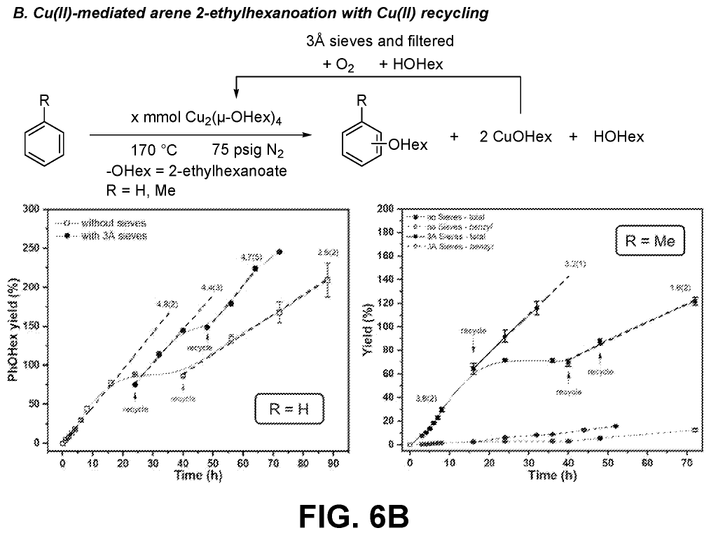

Isolation of products and recycling of Cu(II). The reaction using Cu(OHex)₂ was scaled to 1.1 g where 78% isolated yield of PhOHex and 83% recovery of the Cu(OHex)₂ were achieved (FIG. 6A). We demonstrated recycling of the recovered Cu(OHex)₂ using toluene as the substrate, and no difference in tolyl-to-benzyl selectivity was observed upon use of the recycled Cu(OHex)₂. PhOAc can also be isolated with 69% yield along with 93% recovery of the used Cu(OAc)₂. With knowledge of the effect of molecular sieves, water and O₂, we pursued a demonstration that the Cu(II) salt could be recovered and recycled after a completed reaction, similar in some ways to one version of the commercialized Wacker process (32-35). To do so, after each reaction, 0.5 equivalents of HOHex was added to the reaction solution and stirred under air until the solution turned dark blue indicating that Cu(I) is oxidized to Cu(II). Then 3 Å molecular sieves were added to remove the generated water that was formed from the regeneration of Cu(OHex)₂. After filtration to remove the sieves and trapped water, Cu(OHex)₂ was isolated and reused for the reaction under anaerobic conditions and demonstrated a similar rate as before either using benzene or toluene as the substrate (FIG. 6B), and 2.5 TOs (based on bis-Cu not monomeric Cu) of PhOHex can be achieved. The hydrolysis step of the phenyl carboxylate products was performed using a previously reported procedure (46). In addition, phenol can be formed in >95% yield by reacting MeOH with phenyl carboxylate product, so that the formed methyl ester and remaining MeOH can be readily isolated from the reaction mixture by distillation. Overall, in the recycle experiments, 240% total yield from benzene to phenol was achieved {based on Cu(II)} with 0.6% total conversion of the initial benzene. By changing the molar ratio of benzene and Cu(II) salts along with using solvent, 2.4% conversion of benzene can be reached, however, there is likely a thermodynamic inhibition to achieving higher conversion.

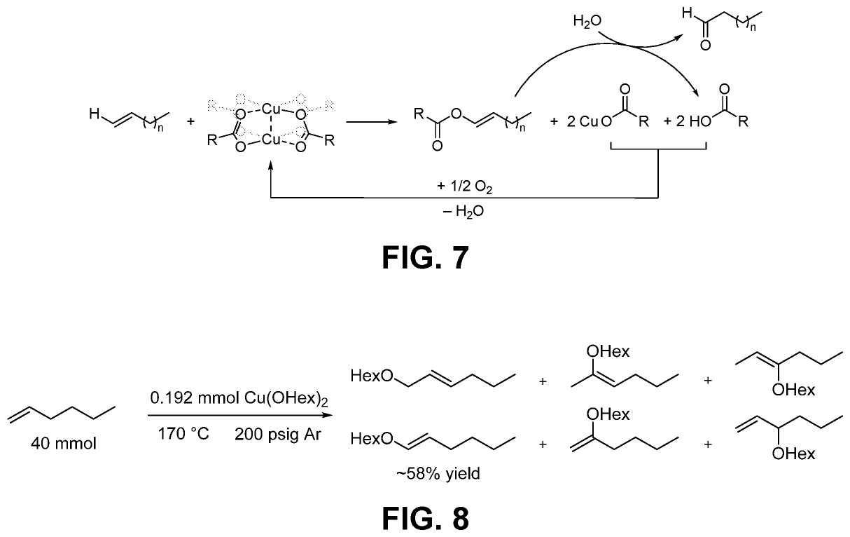

Development of non-directed Cu(II)-mediated C—H acetoxylation of alkenes. Based on the results from the C—H functionalization of benzene and toluene using Cu(II) carboxylate, we thought to extend the study to terminal alkene which could potentially lead to a new synthetic route for long-chain aliphatic aldehyde production as shown in FIG. 7.

By heating 5 mL of a 1-hexene mixture with pre-dried Cu(OHex)₂ (0.192 mmol, 0.48 mol % relative to 1-hexane) to 170° C. under argon (200 psig), hexenyl 2-ethylhexanoates were observed as products based on the GC-MS analysis, in which 1-hexen-1-yl 2-ethylhexanoates were found to be the major product with ˜58% yield relative to the limiting Cu(II) reagent based on ¹H NMR analysis of the reaction solution using peak integration compared to 1-hexene (FIG. 8). Cu(OPiv)₂ and Cu(OAc)₂ have also been examined for the reaction using 1-hexene as the substrate, and similar reactivity was observed as Cu(OHex)₂.

### CONCLUSION

We report a process using copper (II) carboxylate salts to oxidize benzene and toluene without installation of a directing group through a proposed organometallic Cu(II)-mediated C—H activation pathway. While Cu-mediated C—H activation to form intermediates with Cu—C bonds have been proposed previously (13,15), to our knowledge, evidence for non-radical Cu-mediated functionalization of hydrocarbons with strong C—H bonds is rare. Further, we have demonstrated that the copper (II) carboxylate salts used for the reaction can be isolated, recycled, and reused for the reaction with little change in reactivity, which is similar to one version of the commercial Wacker process for ethylene oxidation (32-35). Regeneration of the copper (II) carboxylate salt is achieved in solution using O₂ after each reaction, and with the removal of the generated water, the reaction maintains a similar rate using recycle Cu(II). By adding a hydrolysis step of the phenyl carboxylate product, which is a known reaction (46), this process could provide a coproduct-free synthetic route for phenol production using cheap air-recyclable Cu(II) salts.

Our mechanistic studies demonstrate that the dimeric Cu(II) structure of Cu₂(μ-OAc)₄ lowers the activation barrier for the product forming C—O reductive coupling step from the Cu(III)—Ar intermediate. This proposal shares some similarities to our previously published catalysis for styrene production using benzene and ethylene for which we propose the incorporation of Cu(II) in a Rh/Cu/Rh structure reduces the activation barrier for an O—H bond forming reductive coupling step (47). Thus, the incorporation of a Lewis acidic and oxidizing Cu(II) center (and possibly related metals) into a multi-metallic species might represent a general strategy to facilitate catalytic hydrocarbon oxidation.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. R. J. Schmidt, Industrial catalytic processes-phenol production. Appl. Catal. A-Gen. 280, 89-103 (2005). doi: 10.1016/j.apcata.2004.08.030
2. A. Mancuso, O. Sacco, D. Sannino, et al., One-Step Catalytic or Photocatalytic Oxidation of Benzene to Phenol: Possible Alternative Routes for Phenol Synthesis? Catalysts 10, 1424 (2020). doi: 10.3390/catal10121424
3. K. H. Kuechler, F. M. Benitez Dehydrogenation processes and phenol compositions. U.S. Pat. No. 9,242,918, Jan. 26, 2016.
4. H. Nair, C. L. Becker, R. A. Sothen, et al. Process for producing phenol. U.S. Pat. No. 9,233,890, Jan. 12, 2016.
5. W. D. McGhee Selective introduction of active sites for hydroxylation of benzene. U.S. Pat. No. 5,977,008, Nov. 2, 1999.
6. H. Orita, T. Hayakawa, M. Shimizu, et al., Catalytic hydroxylation of benzene by the copper-ascorbic acid-O2 system. J. Mol. Catal. 42, 99-103 (1987). doi: 10.1016/0304-5102(87)85043-5
7. T. Kitamura, H. Kanzaki, R. Hamada, et al., Liquid-phase oxidation of benzene to phenol by copper catalysts in aqueous solvent with a high acetic acid concentration. Can. J. Chem. 82, 1597-1605 (2004). doi: 10.1139/v04-123
8. A. Häusser, M. Trautmann, E. Roduner, Spin trapping of hydroxyl radicals on Cu/HY zeolites suspended in aqueous solution. Chem. Commun. 47, 6954-6956 (2011). doi: 10.1039/C1CC11181G
9. D. Wang, J. N. Jaworski, S. S. Stahl, Pd-Catalyzed Aerobic Oxidation Reactions: Industrial Applications and New Developments. in Liquid Phase Aerobic Oxidation Catalysis: Industrial Applications and Academic Perspectives, Stahl, S. S.; Alsters, P. L., Eds. (Wiley-VCH, Weinheim, 2016), pp 113-138.
10. S. R. Neufeldt, M. S. Sanford, Controlling Site Selectivity in Palladium-Catalyzed C—H Bond Functionalization. Acc. Chem. Res. 45, 936-946 (2012). doi: 10.1021/ar300014f
11. T. Yoneyama, R. H. Crabtree, Pd(II) catalyzed acetoxylation of arenes with iodosyl acetate. J. Mol. Catal. A: Chem. 108, 35-40 (1996). doi: 10.1016/1381-1169(95)00289-8
12. X. Chen, X.-S. Hao, C. E. Goodhue, et al., Cu(II)-Catalyzed Functionalizations of Aryl C—H Bonds Using O2 as an Oxidant. J. Am. Chem. Soc. 128, 6790-6791 (2006). doi: 10.1021/ja061715q
13. X. Li, Y.-H. Liu, W.-J. Gu, et al., Copper-Mediated Hydroxylation of Arenes and Heteroarenes Directed by a Removable Bidentate Auxiliary. Org. Lett. 16, 3904-3907 (2014). doi: 10.1021/ol5016064
14. B. K. Singh, R. Jana, Ligand-Enabled, Copper-Promoted Regio- and Chemoselective Hydroxylation of Arenes, Aryl Halides, and Aryl Methyl Ethers. J. Org. Chem. 81, 831-841 (2016). doi: 10.1021/acs.joc.5b02302
15. S.-Z. Sun, M. Shang, H.-L. Wang, et al., Cu(II)-Mediated C(sp2)-H Hydroxylation. J. Org. Chem. 80, 8843-8848 (2015). doi: 10.1021/acs.joc.5b01351
16. A. M. Suess, M. Z. Ertem, C. J. Cramer, et al., Divergence between Organometallic and Single-Electron-Transfer Mechanisms in Copper (II)-Mediated Aerobic C—H Oxidation. J. Am. Chem. Soc. 135, 9797-9804 (2013). doi: 10.1021/ja4026424
17. J. Gallardo-Donaire, R. Martin, Cu-Catalyzed Mild C(sp2)-H Functionalization Assisted by Carboxylic Acids en Route to Hydroxylated Arenes. J. Am. Chem. Soc. 135, 9350-9353 (2013). doi: 10.1021/ja4047894
18. W. Wang, F. Luo, S. Zhang, et al., Copper (II)-Catalyzed Ortho-Acyloxylation of the 2-Arylpyridines sp2 C—H Bonds with Anhydrides, Using O₂ as Terminal Oxidant. J. Org. Chem. 75, 2415-2418 (2010). doi: 10.1021/jo1000719
19. M. Shang, Q. Shao, S.-Z. Sun, et al., Identification of monodentate oxazoline as a ligand for copper-promoted ortho-C—H hydroxylation and amination. Chem. Sci. 8, 1469-1473 (2017). doi: 10.1039/C6SC03383K
20. X. Wu, Y. Zhao, H. Ge, Pyridine-enabled copper-promoted cross dehydrogenative coupling of C(sp2)-H and unactivated C(sp3)-H bonds. Chem. Sci. 6, 5978-5983 (2015). doi: 10.1039/C5SC02143J
21. X. Zeng, W. Yan, M. Paeth, et al., Copper-Catalyzed, Chloroamide-Directed Benzylic C—H Difluoromethylation. J. Am. Chem. Soc. 141, 19941-19949 (2019). doi: 10.1021/jacs.9b11549
22. D. K. Pandey, A. B. Shabade, B. Punji, Copper-Catalyzed Direct Arylation of Indoles and Related (Hetero) arenes: A Ligandless and Solvent-free Approach. Adv. Synth. Catal., (2020). doi: 10.1002/adsc.202000312
23. R. Odani, K. Hirano, T. Satoh, et al., Copper-Mediated Formally Dehydrative Biaryl Coupling of Azine N-Oxides and Oxazoles. J. Org. Chem. 80, 2384-2391 (2015). doi: 10.1021/acs.joc.5b00037
24. M. Nishino, K. Hirano, T. Satoh, et al., Copper-Mediated and Copper-Catalyzed Cross-Coupling of Indoles and 1,3-Azoles: Double C—H Activation. Angew. Chem. Int. Ed. 51, 6993-6997 (2012). doi: 10.1002/anie.201201491
25. K. Takamatsu, Y. Hayashi, S. Kawauchi, et al., Copper-Catalyzed Regioselective C—H Amination of Phenol Derivatives with Assistance of Phenanthroline-Based Bidentate Auxiliary. ACS Catal. 9, 5336-5344 (2019). doi: 10.1021/acscatal.9b01145
26. M. Shang, S.-Z. Sun, H.-X. Dai, et al., Cu(OAc)2-Catalyzed Coupling of Aromatic C—H Bonds with Arylboron Reagents. Org. Lett. 16, 5666-5669 (2014). doi: 10.1021/015027377
27. C. Yamamoto, K. Takamatsu, K. Hirano, et al., Copper-Catalyzed Intramolecular Benzylic C—H Amination for the Synthesis of Isoindolinones. J. Org. Chem. 81, 7675-7684 (2016). doi: 10.1021/acs.joc.6b01393
28. M. Shang, S.-Z. Sun, H.-L. Wang, et al., Exceedingly Fast Copper (II)-Promoted ortho C—H Trifluoromethylation of Arenes using TMSCF3. Angew. Chem. Int. Ed. 53, 10439-10442 (2014). doi: 10.1002/anie.201404822
29 X. Wu, J. Miao, Y. Li, et al., Copper-promoted site-selective carbonylation of sp3 and sp2 C—H bonds with nitromethane. Chem. Sci. 7, 5260-5264 (2016). doi: 10.1039/C₆SC01087C
30. R. Wang, Y. Li, R.-X. Jin, et al., Copper-catalyzed oxidative C(sp3)-H/C(sp2)-H cross-coupling en route to carbocyclic rings. Chem. Sci. 8, 3838-3842 (2017). doi: 10.1039/C7SC00250E
31. J. Wang, R. Sang, X. Chong, et al., Copper-catalyzed radical cascade oxyalkylation of olefinic amides with simple alkanes: highly efficient access to benzoxazines. Chem. Commun. 53, 7961-7964 (2017). doi: 10.1039/C7CC04213B
32. J. A. Keith, P. M. Henry, The Mechanism of the Wacker Reaction: A Tale of Two Hydroxypalladations. Angew. Chem. Int. Ed. 48, 9038-9049 (2009). doi: 10.1002/anie.200902194
33. J. Smidt, W. Hafner, R. Jira, et al., Katalytische Umsetzungen von Olefinen an Platinmetall-Verbindungen Das Consortium-Verfahren zur Herstellung von Acetaldehyd. Angew. Chem. 71, 176-182 (1959). doi: 10.1002/ange. 19590710503
34. G. F. M. Eckert, R. Jira, H. M. Bolt, K. Golka, in Ullmann's Encyclopedia of Industrial Chemistry, (Wiley-VCH Verlag, Weinheim, Germany, 2000), pp 1-17.
35. Hoechst Reveals Wacker Process Details. Chem. Eng. News Archive 39, 52-55 (1961). doi: 10.1021/cen-v039n016.p052
36. J. Chen, R. J. Nielsen, W. A. Goddard, et al., Catalytic Synthesis of Superlinear Alkenyl Arenes Using a Rh(I) Catalyst Supported by a “Capping Arene” Ligand: Access to Aerobic Catalysis. J. Am. Chem. Soc. 140, 17007-17018 (2018). doi: 10.1021/jacs.8b07728
37. F. Kong, S. Gu, C. Liu, et al., Effects of Additives on Catalytic Arene C—H Activation: Study of Rh Catalysts Supported by Bis-phosphine Pincer Ligands. Organometallics 39, 3918-3935 (2020). doi: 10.1021/acs.organomet.0c00623
38. D. Munz, M. Webster-Gardiner, R. Fu, et al., Proton or Metal? The H/D Exchange of Arenes in Acidic Solvents. ACS Catal. 5, 769-775 (2015). doi: 10.1021/cs501620f
39. C. E. Elwell, N. L. Gagnon, B. D. Neisen, et al., Copper-Oxygen Complexes Revisited: Structures, Spectroscopy, and Reactivity. Chem. Rev. 117, 2059-2107 (2017). doi: 10.1021/acs.chemrev.6b00636
40. L. M. Mirica, X. Ottenwaelder, T. D. P. Stack, Structure and Spectroscopy of Copper-Dioxygen Complexes. Chem. Rev. 104, 1013-1046 (2004). doi: 10.1021/cr020632z
41. E. A. Lewis, W. B. Tolman, Reactivity of Dioxygen-Copper Systems. Chem. Rev. 104, 1047-1076 (2004). doi: 10.1021/cr020633r
42. A. Conde, L. Vilella, D. Balcells, et al., Introducing Copper as Catalyst for Oxidative Alkane Dehydrogenation. J. Am. Chem. Soc. 135, 3887-3896 (2013). doi: 10.1021/ja310866k
43. P. Sharrock, M. Melnik, Copper (II) acetates: from dimer to monomer. Can. J. Chem. 63, 52-56 (1985). doi: 10.1139/v85-009
44. E. M. Simmons, J. F. Hartwig, On the Interpretation of Deuterium Kinetic Isotope Effects in C—H Bond Functionalizations by Transition-Metal Complexes. Angew. Chem. Int. Ed. 51, 3066-3072 (2012). doi: 10.1002/anie.201107334
45. W. D. Jones, Isotope Effects in C—H Bond Activation Reactions by Transition Metals. Acc. Chem. Res. 36, 140-146 (2003). doi: 10.1021/ar020148i
46. W. Medina-Ramos, M. A. Mojica, E. D. Cope, et al., Water at elevated temperatures (WET): reactant, catalyst, and solvent in the selective hydrolysis of protecting groups. Green Chem. 16, 2147-2155 (2014). doi: 10.1039/C₃GC42569J
47. C. B. Musgrave, W. Zhu, N. Coutard, et al., Mechanistic Studies of Styrene Production from Benzene and Ethylene Using [(η2-C2H4)2Rh(μ-OAc)]2 as Catalyst Precursor: Identification of a Bis-Rhl Mono-Cull Complex As the Catalyst. ACS Catal. 11, 5688-5702 (2021). doi: 10.1021/acscatal.1c01203

1. A method for producing an aryl ester or a vinyl ester, the method comprising reacting an aromatic compound with CuX₂ to produce the aryl ester or reacting an alkene with CuX₂ to produce the vinyl ester, wherein X is a carboxylate group having the formula 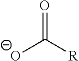
wherein R is an alkyl group.

2. The method of claim 1, wherein the aromatic compound is benzene or substituted benzene.

3. The method of claim 1, wherein the aromatic compound is benzene substituted with one or more alkyl groups.

4. The method of claim 1, wherein the alkene is a terminal alkene.

5. The method of claim 1, wherein the alkene is a branched or straight chain C₂ to C₂₄ alkene.

6. The method of claim 1, wherein the vinyl ester is a terminal vinyl ester.

7. The method of claim 1, wherein R is a branched or straight chain C₁ to C₂₀ alkyl group.

8. The method of claim 1, wherein R is —CH₃, —CH(CH₃)₂, —C(CH₃)₃, or —(CH₂)₅CH₃ or 2-ethylthexanoate.

9. The method of claim 1, wherein the reaction is conducted under an inert atmosphere.

10. The method of claim 1, wherein CuX₂ is from about 0.1 mol % to about 10.0 mol % relative to the molar amount of the aromatic compound or alkene, wherein the sum of the molar amount of the aromatic compound or alkene and CuX₂ is 100 mol %.

11. The method of claim 1, wherein the reaction comprises admixing the aromatic compound or alkene with CuX₂ at a temperature of from about 150° C. to about 300° C.

12. The method of claim 1, wherein CuX₂ is anhydrous.

13. The method of claim 1, wherein the reaction is conducted in an organic solvent.

14. The method of claim 13, wherein the organic solvent is an anhydrous solvent.

15. The method of claim 1, wherein after the reaction CuX is produced, wherein CuX is converted to CuX₂ by (1) adding RC(O)OH in the presence of oxygen to the reaction to produce CuX₂ and water, wherein R is the same alkyl group as CuX₂, and (2) removing water from the reaction.

16. The method of claim 15, wherein water is removed by molecular sieves or distillation.

17. The method of claim 15, wherein the recycled CuX₂ is reintroduced into the reaction with the aromatic compound or alkene.
